(19) United States
(12) Patent Application Publication
(10) Pub. No.: US 2025/0332236 A1
(43) Pub. Date: Oct. 30, 2025

(54) CANCER IMMUNOTHERAPY ORAL VACCINE

(71) Applicants: Jovelle Laoag Fernandez, Henderson, NV (US); Alex Marcelo Fernandez, Henderson, NV (US)

(72) Inventors: Jovelle Laoag Fernandez, Henderson, NV (US); Alex Marcelo Fernandez, Henderson, NV (US)

(21) Appl. No.: 18/975,316

(22) Filed: Dec. 10, 2024

Related U.S. Application Data
(60) Provisional application No. 63/608,381, filed on Dec. 11, 2023.

(51) Int. Cl. A61K 39/00

(52) U.S. Cl. CPC A61K 39/0011; A61K 2039/523; A61K 2039/542

(57) ABSTRACT

A method of producing an immunotherapeutic mucosal cancer vaccine which is orally administered includes amplifying and synthesizing a cancer proliferating cell nuclear antigen (caPCNA); fusing enzymatically the caPCNA with an anchoring domain gene to produce a fusion gene; cloning the fusion gene into an expression vector; transforming the expression vector into Lactococcus lactis (L. lactis) cells to produce a transformant; harvesting the transformant; and encapsulating the transformant. The transformant exhibits display of the caPCNA on the cell surface of L. lactis. An immunotherapeutic mucosal vaccine composition contains the orally administered vaccine to treat various types of cancer that are positive to the tumor antigen, caPCNA. A method of immunotherapy for a mucosal vaccine to treat cancer includes orally administering to a subject an amount of the immunotherapeutic mucosal vaccine composition. The immunotherapy composition triggers targeted killing of cancer cells by stimulating immune cells to treat various cancers.

### CROSS-REFERENCE TO RELATED APPLICATION

This application claims the benefit of priority of U.S. provisional application No. 63/608,381 filed Dec. 11, 2023, the contents of which are herein incorporated by reference.

### REFERENCE TO SEQUENCE LISTING XML SUBMITTED ELECTRONICALLY

This application contains a Sequence Listing which has been submitted in XML format via PatentCenter and is hereby incorporated by reference in its entirety. The Sequence Listing XML file, created Nov. 30, 2024, is named “577810038np Sequence Listing” and has a size of 2,203 bytes.

### BACKGROUND OF THE INVENTION

The present invention relates to immunotherapeutic treatment of cancer and, more particularly, to an immunotherapeutic mucosal vaccine for cancer, administered orally.

Our mission is to advance the health and welfare of the American public, as described in the American Innovation and Competitiveness Act Broader Impacts Review Criterion Update. This innovation will help 4 out of 5 Americans who do not experience an optimal response from current advanced cancer antigen-positive treatments (Chen et al., 2019) by providing a novel, safe, and effective cancer treatment that will result in improved quality of life.

One out of five United States (US) deaths are caused by cancer. For every 100,000 people, 403 new cancer cases were reported and 144 people died of cancer (US Cancer Statistics, 2020). An estimated 1,958,320 million new cases are expected to be diagnosed, with an associated 609,820 cancer deaths in the US in 2023 (American Cancer Society [ACS], 2023). The incidence rate for cancer globally is expected to increase to >30 M new cases/year by 2040, with expected deaths of >16 M (International Agency for Research on Cancer, 2021). Cancer death rates are higher among minority groups and poorer regions (Islami, 2022).

Currently available cancer treatment is inadequate. Despite advances in cancer drug development, existing treatments produce only up to 30% clinically acceptable therapeutic responses, and many have dangerous or lethal side effects. Administration of such options, a majority of which require clinic visits, hospitalization, and trained personnel, burdens the patients, their families, caregivers, providers, and payers.

There are many routes that caregivers and patients pursue in the fight against cancer. Surgical options, which pose risks such as damage to other organs, require highly skilled surgeons and hospitalizations. Radiation therapy (such as that provided by General Electric [GE] Healthcare and Siemens) involves expensive equipment and specialized staff training. Patients face risks of damage to normal tissues, dose delivery, body positioning errors, and severe adverse events such as sterility (Knight E, et al., 2021). Chemotherapy (provided by Roche, Novartis and others) requires hospital or clinical facilities with trained staff. Severe adverse events include cardiomyopathy and neuropathy (Behranvand N. et al, 2022). Companies including AbbVie and Genentech, offer expensive targeted therapy (with side effects including hypertension and bleeding) that may need to be administered in a hospital or clinical facility (Xie Y, et al., 2020). Eli Lilly, Pfizer, and others offer hormonal therapy, but it may lead to severe adverse effects (Krauss K et al., 2020) such as thromboembolic disease, and other cancers such as endometrial cancer.

Current state-of-the-art processes leading this field include immunotherapy and precision-based oncology, but they offer limited benefits (Bagchi et al., 2022), are expensive at >$400 K/year/person (Rimer B, 2018), and have restricted accessibility (ACS, 2020). Many patients who receive immunotherapy such as those from Merck and Bristol Myers Squibb show hyperprogression of cancer (Frelaut M, et al., 2019) with about a third responding at the beginning but ultimately developing tumor resistance (Dubosz P et al, 2022). Recipients also face immune-related adverse events and some lead to emergency room (ER) visits, with 25% related to one or more immune-related events (El Majzoub, I. et al, 2019).

To date, the most successful cancer therapies involve immune checkpoint inhibitors. However, these treatments are associated with immune-related toxicities and cancer resistance, and most patients continue to have disease progression despite treatment (Dobosz et al., 2022). The discovery of alternative approaches requires unique skillsets and knowledge of stimulating additional checkpoint pathways using effective cancer-specific presentation and delivery of tumor-associated antigens (TAA). Ongoing TAA-based cancer vaccine studies on injectable treatments with suboptimal peptide presentation, restricted immune response stimulation and killing of cancer cells, and limited clinical application have been pursued. An acceptable treatment response can only be achieved with more frequent and/or incremental dosing, which has associated safety concerns (Malonis et al, 2019). Another TAA-based vaccine, while orally administered, utilizes long amino acid sequences of Wilm's tumor 1 (WT1) protein, which lead to an expensive and tedious manufacturing process (Kitagawa et al., 2017). In addition, WT1 has been shown to be cancer-causing (Oji Y et al, 1999).

Challenges exist with complex interventions of precision-based oncology such as chimeric antigen receptor (CAR)-T, including severe life-threatening toxicity, modest anti-tumor activity, and limited tumor infiltration (Sterner et al, 2021). Chimeric antigen receptor T (CAR-T) treatments from Celgene and Johnson & Johnson (among others) are very expensive and require special manufacturing techniques. Side effects include cytokine-release syndrome with fever and hypotension (Santomasso B. et al., 2019). CAR-T needs improved cell engineering and genome-editing technologies (Muthukutty et al., 2023).

As can be seen, there is a need for a safe, effective, affordable, and accessible cancer treatment that can be administered orally, giving cancer patients control of their treatment. In particular, there is a need to stimulate additional immune checkpoint pathways with effective, cancer-specific presentation and delivery of a definitive TAA with broad spectrum therapeutic agent for cancer treatment. An accessible and affordable cancer treatment including a simple, effective, and safe presentation and delivery system for TAA can achieve improved outcomes.

This is an important issue to tackle given that current trends to support human health impact the human gut microbiome. Human microbiomes have been collectively considered as an organ per se, with roughly 10× the number of microbial cells compared with human cells in the body, amounting to 100 trillion microbes in the body, yet weighing only 2 kilograms, implicating their critical role in disease development, including cancer. This aims to provide further knowledge on the value of good microbiomes and to utilize what we have in nature to treat diseases with activation of our largest immune defense, which is the gut, to combat cancer.

### SUMMARY OF THE INVENTION

In one aspect of the present invention, a method of producing an immunotherapeutic mucosal cancer vaccine administered orally comprises cancer proliferating cell nuclear antigen (caPCNA), a broad-spectrum therapeutic agent for cancer will serve as the TAA gene into a plasmid using a protein anchor to the cell wall of a selected microorganism, which can be genetically modified and non-pathogenic Lactococcus lactis (L. lactis) through sortase-mediated covalent anchoring followed by fusion. This formulation is referred to herein as L. lactis with surface display of caPCNA, or LlacP for short. Optimal anchoring domain to covalently bind surface attachment of the caPCNA is achieved using LPXTG motif-containing protein and fusion gene usp45 signal peptide or gene (SP310, SP310mut or SP310mut2) on the cell surface of the L. lactis. The fusion gene is cloned into a suitable expression vector, with a strong promoter, a ribosome binding site, a terminator, an origin of replication, and a selectable marker. Transformants are produced using electroporation, conjugation or sonoporation for caPCNA expression and surface display on the cell wall of L. lactis. Transformants are assessed and screened for caPCNA expression and surface display. Selection markers include antibiotic resistance or reporter genes. Screening methods utilize Western blotting or flow cytometry. Optimization of LlacP is determined via various culture conditions and induction parameters for caPCNA production and display. Variable factors such as temperature, pH, media composition, inducer concentration, and induction time. For the final product, cells are separated from the culture supernatant using centrifugation or filtration and purified using chromatography or precipitation. Polymer coating with calcium alginate with protamine composite as a shell with double-coating is used to encapsulate LlacP. The efficacy of microencapsulation is determined by decapsulating while assessing the viability at 0,30 and 60 days after encapsulation.

In another aspect of the present invention, an immunotherapy composition comprises the mucosal cancer vaccine, an orally administered vaccine produced by the method.

In another aspect of the present invention, a method of immunotherapy for a cancer comprises administering orally to a subject an effective amount of the immunotherapeutic mucosal vaccine composition.

LlacP will display caPCNA on the engineered L. Lactis using secretory fusion protein and anchored on L. lactis cell surface. LlacP utilizes the gut's immune system, which has a surface area of 32 sqm. When swallowed, caPCNA is presented to the dendritic cells (DC) scattered in the gut to prime and activate the immune response. From the Peyer's patches, DC move to the mesenteric lymph nodes and induce CTA-specific CD8 cytotoxic-T cells and CD4 helper-T cells. Cytotoxic-T cells infiltrate and kill the tumor cells displaying caPCNA, supported by helper-T cells. L. lactis enhances altered antigen presentation machinery contributing to immune suppression, tumor progression and metastasis in advanced caPCNA-positive cancers such as colorectal, breast, prostate, ovarian, lung, cervical, brain and liver cancers.

LlacP addresses concerns of efficacy and treatment adherence associated with current cancer treatments. LlacP is easily stored and administered, is affordable, and delivers better outcomes. LlacP can decrease cancer rates by 30%, increase treatment adherence by 80%, and reduce treatment costs by 50%. LlacP can help 4 out of 5 Americans who do not experience an optimal response from current cancer treatments by providing a novel, safe, and effective cancer treatment, resulting in improved quality of life. LlacP offers a significant beneficial impact on society by improving cancer treatment, increasing awareness of cancer management, and providing greater autonomy to cancer patients to manage their health, reducing the economic burden of cancer on all individuals and society while helping push forward the 2022 federal Cancer Moonshot objective of addressing progress against cancer. LlacP improves survival rates, cancer regression, and quality of life among patients with caPCNA-positive cancers, such as colorectal, breast, prostate, ovarian, lung, cervical, brain and liver.

Minority groups and poorer regions, where deaths from cancer are more frequent, benefit disproportionately.

LlacP helps prevent cellular dysplasia and carcinogenesis and decreases susceptibility to certain cancers, benefiting healthy individuals as well.

Newly diagnosed cancer patients and those on treatment regimens (chemo-or immunotherapy) can benefit from successful immune surveillance for optimal cancer treatment and regression via mediation of CD8+ T cells with cross-reactive T-cell epitopes present in microbiome, such as L. lactis, and cancer. In combination, less dosing of chemo-or immunotherapy results in diminished safety concerns, improved treatment adherence, and better outcomes. The microbiome's role in tumor microenvironment moderates response and toxicity to traditional chemo-and immunotherapy. In both instances, patient outcomes are favorably affected.

These and other features, aspects and advantages of the present invention will become better understood with reference to the following drawings, description, and claims.

### BRIEF DESCRIPTION OF THE DRAWINGS

FIG. 1 is a schematic diagram of a microorganism for use in the encapsulated and orally administered immunotherapeutic mucosal cancer vaccine composition of the present invention;

FIG. 2A is a flowchart of a method of producing an orally administered immunotherapeutic mucosal cancer vaccine according to an embodiment of the present invention;

FIG. 2B is a continuation of the flowchart of FIG. 2A; and

FIG. 3 is a flowchart of a method of treating a cancer patient according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is immunotherapeutic mucosal vaccine composition for oral administration to treat various types of cancer.

The composition discussed herein is sometimes referred to as “LlacP”. LlacP is the world's first immunotherapeutic mucosal cancer vaccine using L. lactis delivery of caPCNA, thereby leveraging the microorganism's probiotic cancer health benefits. The immunotherapeutic mucosal cancer vaccine can be made using a human gut microorganism, L. lactis, which is genetically modified to express and surface display the TAA, caPCNA. The mucosal vaccine utilizes the gut's immune system, which has a surface area of 32 square meters (sqm). The immunotherapeutic mucosal cancer vaccine may stimulate multiple immune responses in the gut and circulation to optimize the targeted destruction of existing and developing cancer cells without affecting non-cancerous cells. Without being bound by theory, it is anticipated that all caPCNA-positive cancers may respond to the immunotherapeutic mucosal cancer vaccine composition disclosed herein.

The Active Pharmaceutical Ingredients (API) generally include a microorganism, L. lactis, and TAA (caPCNA). Optimal logistics for delivery and storage of the API are developed, accommodating biosafety regulations and standards for a genetically modified generally regarded as safe microorganism.

The microbiome is not particularly limited, as long as it has anti-cancer effects. In a non-limiting example, the microbiome may comprise the genetically modifiable microorganism Lactococcus lactis (L. lactis). The TAA, which is present in various types of cancer, generally only induces an immune response in the tumor microenvironment of the cancer cells, which express the specific cancer-associated antigen, such as cancer proliferating cell nuclear antigen (caPCNA). For example, the immunotherapeutic mucosal cancer vaccine composition may include caPCNA displayed on the L. lactis cell surface.

In embodiments, the TAA is cancer Proliferating Cell Nuclear Antigen (caPCNA). Specifically, caPCNA demonstrates antigenic relevance, which leads to functional recognition by pre-existing T lymphocytes that recognize microbial peptides. CaPCNA is introduced to the mucosal immune system of the intestines, with inductor sites located in the Peyer's Patches (PP). This activates the involvement of local secretory antibody responses (CD4+ and CD8+ T cells). Antigen-specific B-cell and T-cell responses are initiated with mediation of the effector sites. Selective expression factors on B and T cells facilitate the connection of inductor and effector sites. Regarding activation, antigen presenting cells (APCs) such as DCs, are engaged with absorbed caPCNA and contribute to effector responses with recruitment and activation of T and B cells. CaPCNA sampling and trafficking occur via induction of immunoglobulins from mucosal DCs and microfold cells located in PP. Cross-priming of cytotoxic T lymphocytes and imprinting mucosal homing receptors lead to anti-tumor functionality. T cells and APCs migrate from the gut to the mesenteric lymph nodes, tumors and their lymph nodes. For enhancement, L. lactis achieves adjuvanticity and modulate immune tumor control due to microbiome-associated factors that imprint both effector and suppressive arms of immunity. Locally primed immune cells seed distantly with caPCNA-specific effector/memory phenotypes to protect against any future tumor. This treatment contains a sparing effect. With specific caPCNA expression only in cancer cells, induction of the immune response only occurs in their tumor microenvironments while sparing normal cells.

Newly diagnosed cancer patients and those on treatment regimens (chemo-or immunotherapy) will benefit from successful immune surveillance for optimal metastatic cancer treatment and regression. Combination of antigenicity and adjuvanticity is critical for cancer cells to be susceptible to immune surveillance and LlacP provides mucosal vaccine cancer treatment with immune surveillance achieved by accomplishing both antigenicity and adjuvanticity from the caPCNA and L. lactis, respectively. The immune mechanism leads to elimination of malignancies by recognizing cells that express caPCNA, leading to potent immune surveillance.

Healthy individuals are exposed to inflammatory changes that drive cancer development. Immune cells that prevent cancer need to be recruited. This will lead to cancer cell apoptosis and prevent cancer from occurring. With LlacP's penetration to the gut, secretion of metabolites results in an indirect immune response against cancer growth with modulation of immune system via several mechanisms. Lipopolysaccharides activate toll-like receptor-4 that further activates T-cell-mediated immune responses against cancer. Short-chain fatty acids, such as butyrate and propionate, are produced and inhibit histone deacetylases of cancer cells, resulting in apoptosis.

In some embodiments, LlacP may be tailored to the patient's unique microbiota or cancer genetic profile to enhance therapeutic effectiveness. For example, another safe microorganism may be used in conjunction with LlacP and/or another TAA may be added to the L. lactis.

Amplification and synthesis of caPCNA gene with desired biophysical properties is determined including the number of residues, isoelectric point, hydrophobicity, net charge at pH7, mass spectrometry, and number of modifications. Genes of caPCNA are obtained by polymerase chain reaction (PCR) or gene synthesis. A plasmid containing caPCNA gene is created using LPXTG motif-containing protein anchor to the cell wall through sortase-mediated covalent anchoring.

Fusion of caPCNA with anchoring domain that is compatible with L. lactis is performed using optimal anchoring domain that covalently binds caPCNA to cell surface of L. lactis. A usp45 signal peptide at the N-terminus of the fusion gene derived from lactococcal usp45 gene (SP310, SP310mut, and SP310mut2) is added. Any suitable anchoring domain can be used to covalently or non-covalently bind TAA to the cell surface of the microorganism. In embodiments, the anchoring protein is the ChW-containing domain of the lactococcal phage AM12 endolysin (cAM12). Fusion gene is cloned into a suitable expression vector for L. lactis. Expression vector and cloning follows. The expression vector has a strong promoter, a ribosome binding site, a terminator, an origin of replication, and a selectable marker. Transformants for caPCNA gene expression are produced with surface display of caPCNA on the cell surface of L. lactis. Transformation procedure of expression vector is performed on L. lactis using electroporation, conjugation or sonoporation.

Appropriate transformants that express and surface display caPCNA gene are identified. This involves assessment of transformants for caPCNA expression and surface display. Antibiotic resistance or reporter genes are used as selection markers. Screening methods use Western blotting or flow cytometry. Optimal caPCNA production and display is determined by investigating culture conditions and induction parameters for caPCNA production and display. Variable factors such as temperature, pH, media composition, inducer concentration, and induction time are utilized.

For the final product, cells from the culture supernatant are separated using centrifugation or filtration and purified using chromatography or precipitation. This results to caPCNA-displaying L. lactis cells that are harvested and purified.

The final purified product is encapsulated, e.g., by layer-by-layer assembly of chitosan and alginate, to maintain stability while promoting the desired immune effect. The process preferably maintains the vitality of the formulation, avoiding dehydration and thermal inactivation. A polymer coating with calcium alginate with protamine composite is used as a shell, produced using extrusion, emulsion, or spray drying methods. The encapsulation method is selected to retain the most viability, thermotolerance, encapsulation yield, and product's biophysical properties. For the encapsulation, an appropriate assembly process is performed with double-coating microencapsulation. The encapsulated product is evaluated for viability, thermotolerance, encapsulation yield, and product's biophysical properties. The efficacy of microencapsulation is evaluated by decapsulating while assessing the viability at 0, 30, and 60 days after encapsulation. The final product is a viable and stable immunotherapeutic mucosal vaccine that effectively delivers caPCNA to caPCNA-positive cancer cells.

Co-administration of the immunotherapeutic mucosal cancer vaccine disclosed herein with chemo-or immunotherapy suppresses cancer growth by shifting systemic immunity. LlacPs' composition which includes caPCNA and L. lactis exhibits both antigenicity and adjuvanticity, respectively, leading to potent immune surveillance, eliminating malignancies by recognizing cells that express caPCNA. L. lactis modulates immune tumor control due to the microorganism-associated factors that imprint both effector and suppressive arms of immunity. Locally primed immune cells seed distantly with caPCNA-specific effector/memory phenotypes to protect against any future tumor. If LlacP is co-administered with chemo-or immunotherapy, there is shifting systemic immunity for immune surveillance and suppression of cancer growth. This occurs via mediation of CD8+ T cells with cross-reactive T-cell epitopes present in L. lactis and cancer. Another pathway is the effect on innate immunity with gut L. lactis resetting of APC functions. In combination, there is less dosing of chemo-or immunotherapy, resulting in diminished safety concerns, improved treatment adherence, and better outcomes.

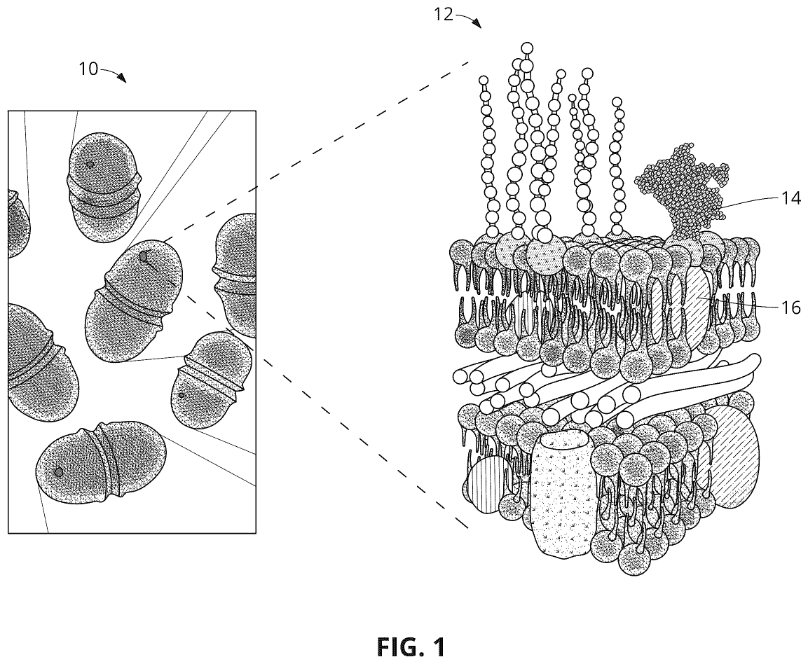

Referring to FIGS. 1-3, FIG. 1 illustrates the microorganism Lactococcus lactis (L. lactis) 10 with surface display of cancer proliferating cell nuclear antigen (caPCNA), an exemplary microorganism for use in the present invention, as well as the magnified cell wall 12 of L. lactis, showing fusion of caPCNA 14 with the anchoring protein 16, in this case ChW-containing domain of the lactococcal phage AM12 endolysin (cAM12).

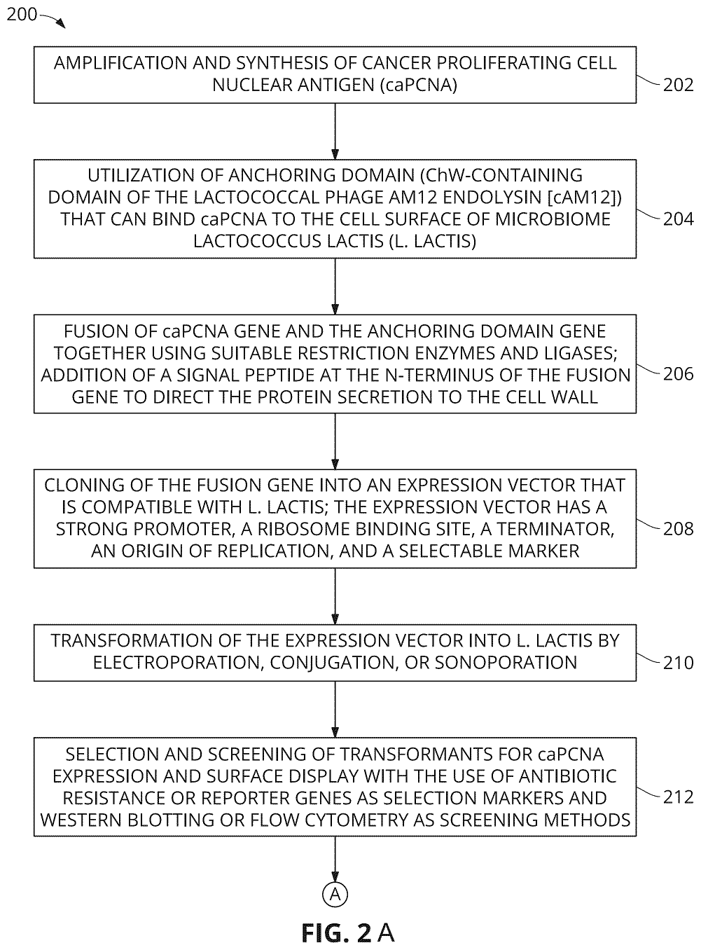

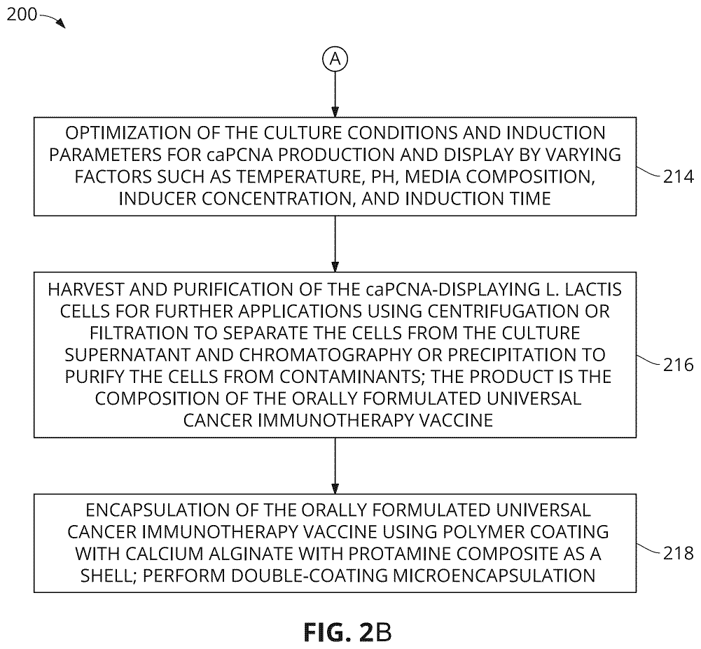

FIGS. 2A and 2B illustrate a method 200 of manufacturing the immunotherapeutic mucosal cancer vaccine according to an embodiment of the present invention. First, the TAA (e.g., caPCNA) is amplified and synthesized 202. A microorganism-compatible plasmid containing the TAA gene and a covalent or non-covalent anchoring domain (e.g., cAM12) 204 is manufactured and fused 206 with the sourced microorganism (e.g., L. Lactis) at the cell surface using restriction enzymes and ligases. Optimal anchoring domain to covalently bind surface attachment of the caPCNA is achieved using LPXTG motif-containing protein and fusion gene usp45 signal peptide or gene (SP310, SP310mut or SP310mut2) on the cell surface of the L. lactis. A signal peptide added at the N-terminus of the fusion gene directs protein secretion to the cell wall. The fusion gene is cloned 208 into a suitable expression vector to develop transformants that express and surface display the TAA, while retaining 100% of TAA's biophysical properties. The transformation procedure 210 of the expression vector into the microorganism uses electroporation, conjugation or sonoporation. The transformants are assessed for TAA expression and surface display, selected using selection markers such as antibiotic resistance or reporter genes, and screened 212 using a method including, e.g., Western blotting or flow cytometry. To ensure the composition's stability, the culture conditions and induction parameters for TAA production and display are optimized 214. Variables such as temperature, pH, media composition, inducer concentration, and induction time are evaluated. The composition is harvested and purified 216 to remove contaminants, separating cells from the culture supernatant using centrifugation or filtration and purifying using chromatography or precipitation. The purified product is encapsulated in a calcium alginate with protamine composite by double-coating microencapsulation 218.

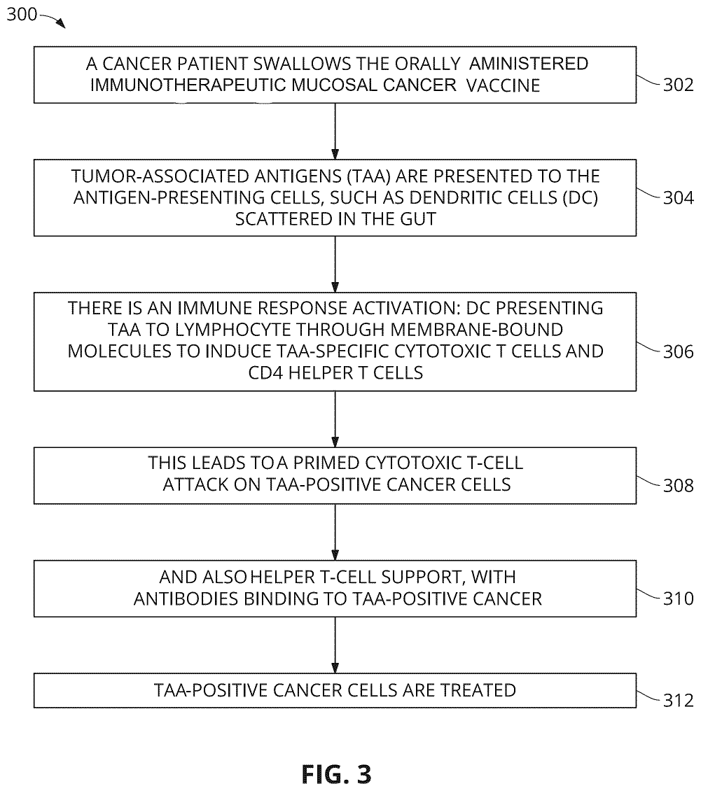

With respect to FIG. 3, the composition is used 300 by swallowing 302 the encapsulated product, thereby presenting the TAA to the dendritic cells (DC) 304 scattered in the gut to prime and activate the immune response. From the Peyer's patches, DC move to the mesenteric lymph nodes and induce TAA-specific CD8cytotoxic-T cells and CD4 helper-T cells 306. Cytotoxic-T cells infiltrate and kill the tumor cells displaying the TAA 308, supported by helper-T cells 310. The latter treats 312 cancer resurgence, including future metastasis. The microorganism further enhances antitumor activity of the immune system.

It should be understood, of course, that the foregoing relates to exemplary embodiments of the invention and that modifications may be made without departing from the spirit and scope of the invention as set forth in the following claims.

What is claimed is:

1. A method of producing an immunotherapeutic mucosal vaccine which is orally administered for various types of cancer, comprising:
amplifying and synthesizing a cancer proliferating cell nuclear antigen (caPCNA);
fusing enzymatically the caPCNA with an anchoring domain gene to produce a fusion gene;
cloning the fusion gene into an expression vector;
transforming the expression vector into Lactococcus lactis (L. lactis) cells to produce a transformant;
harvesting the transformant; and
encapsulating the transformant;
wherein the transformant exhibits display of the caPCNA on the L. lactis cells.

2. The method of claim 1, wherein the transformation comprises a deoxyribonucleic acid (DNA) transfer process selected from the group consisting of: electroporation, conjugation, sonoporation, and any combination thereof.

3. The method of claim 1, wherein the fusion further comprises covalently binding the caPCNA on the L. lactis cells using LPXTG motif-containing protein; and wherein the fusion gene is selected from the group consisting of: Ubiquitin Specific Peptidase 45 (usp45); SP310 or a mutation thereof; and any combination thereof.

4. The method of claim 1, further comprising adding a signal peptide at an N-terminus of the fusion gene before cloning the fusion gene into the expression vector.

5. The method of claim 1, further comprising screening the transformant after transformation by Western blotting and/or flow cytometry.

6. The method of claim 1, wherein the harvesting comprises separating transformed cells from a culture supernatant by centrifugation and/or filtration.

7. The method of claim 6, further comprising purifying the transformed cells of contaminants by chromatography and/or precipitation.

8. The method of claim 1, wherein the encapsulation is performed by a process selected from the group consisting of extrusion, emulsion, spray drying, and any combination thereof.

9. The method of claim 1, wherein the encapsulation produces double coated microcapsules containing the transformant.

10. An immunotherapeutic mucosal vaccine composition comprising the immunotherapeutic mucosal vaccine produced by the method of claim 1.

11. The immunotherapeutic mucosal vaccine composition of claim 10, wherein the immunotherapeutic mucosal vaccine composition is formulated for oral administration.

12. The immunotherapeutic mucosal vaccine composition of claim 10, wherein the immunotherapeutic mucosal vaccine composition has an anti-tumor effect on at least one caPCNA-positive cancer.

13. A method of immunotherapy for a cancer, comprising:
administering orally to a subject the immunotherapeutic mucosal vaccine composition of claim 10 in an amount effective to activate secretory antibody responses in mucosal dendritic cells.

14. The method of claim 13, further comprising co-administering the immunotherapeutic mucosal vaccine composition with chemotherapy or immunotherapy.


SEQUENCE LISTING (1 sequence)

SEQ ID NO: 1 - protein, 261 aa, Homo sapiens
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS